(19) United States
(12) Patent Application Publication
(10) Pub. No.: US 2013/0018385 A1
(43) Pub. Date: Jan. 17, 2013

(54) Polypectomy Snare Device

(71) Applicants: Ken Keene, Winchester, MA (US); Larry Stanton, Burlington, MA (US); Ruth Cheng, Natick, MA (US); Gary Kappel, Acton, MA (US); Gerald R. Heller, Bedford, MA (US)

(72) Inventors: Ken Keene, Winchester, MA (US); Larry Stanton, Burlington, MA (US); Ruth Cheng, Natick, MA (US); Gary Kappel, Acton, MA (US); Gerald R. Heller, Bedford, MA (US)

(73) Assignee: BOSTON SCIENTIFIC SCIMED, INC., Maple Grove, MN (US)

(21) Appl. No.: 13/544,167

(22) Filed: Jul. 9, 2012

Related U.S. Application Data
(60) Provisional application No. 61/506,433, filed on Jul. 11, 2011.

(51) Int. Cl. A61B 17/26

(52) U.S. Cl. 606/113

(57) ABSTRACT

Medical devices are disclosed including polypectomy snare devices. An example polypectomy snare device may include an elongated sheath having a distal end. A hollow shaft may extend through and be movable relative to the sheath. A distal portion of the shaft may have a slit formed therein. The shaft may be shiftable between a first configuration wherein the distal portion is substantially straight and a second configuration wherein the distal portion includes a bend. A snare wire may be disposed within the shaft. A distal section of the snare wire may extend through the slit when the shaft is in the second configuration.

### CROSS-REFERENCE TO RELATED APPLICATION

This application claims priority under 35 U.S.C. §119 to U.S. Provisional Application Ser. No. 61/506,433, filed Jul. 11, 2011, the entirety of which is incorporated herein by reference.

### TECHNICAL FIELD

The present invention pertains to medical devices, and methods for manufacturing medical devices. More particularly, the present invention pertains to polypectomy snare devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

The invention provides design, material, manufacturing method, and use alternatives for medical devices including polypectomy snare devices. An example polypectomy snare device may include a hollow shaft. A distal portion of the shaft may have a slit formed therein. The shaft may be shiftable between a first configuration wherein the distal portion is substantially straight and a second configuration wherein the distal portion includes a bend. A snare wire may be disposed within the shaft. A distal section of the snare wire may extend through the slit when the shaft is in the second configuration.

Another example polypectomy snare instrument may include a hollow shaft may extend through and be movable relative to the sheath. The shaft may have a distal portion and may terminate at a distal end. The distal portion of the shaft may be preformed to have a curvature when the distal portion of the shaft is extended distally beyond the distal end of the sheath. The distal portion of the shaft may have a slit formed therein. A flexible wire may be disposed within the shaft. The wire may be controllably extendible through the longitudinal slit.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a partial cross-sectional side-view cross-section of an example medical device;

FIG. 2 is a side view of the example snare of FIG. 1, with a distal portion of the hollow shaft extended distally beyond a distal end of the sheath;

FIG. 3 is a side view of the example snare of FIGS. 1 and 2, with the distal portion of the hollow shaft extended distally beyond the distal end of the sheath, and with the snare wire distally advanced to bow outward through the slit in the hollow shaft;

FIG. 4 is a side view of an example handle disposed at the proximal end of the example snare of FIGS. 1-3;

FIG. 5 is a top view of an example snare having a snare wire with a semicircular bow;

FIG. 6 is a top view of an example snare having a snare wire with an angled bow; and

FIG. 7 is a top view of an example snare having a snare wire with a bracket-shaped bow.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

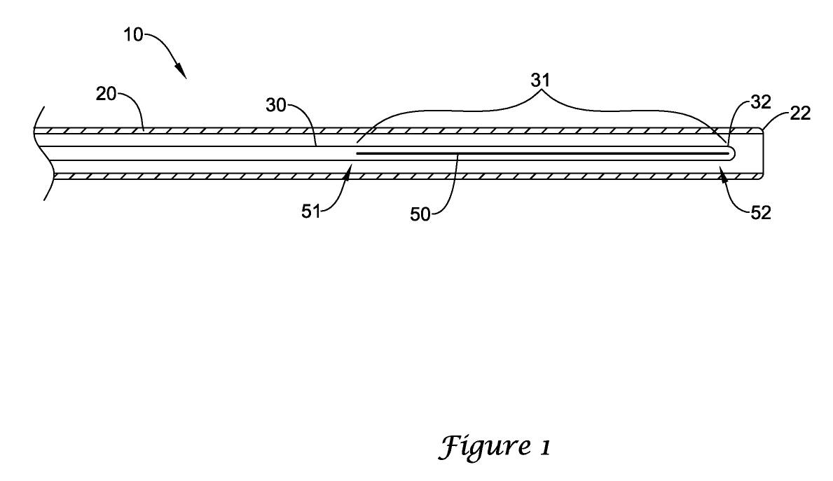

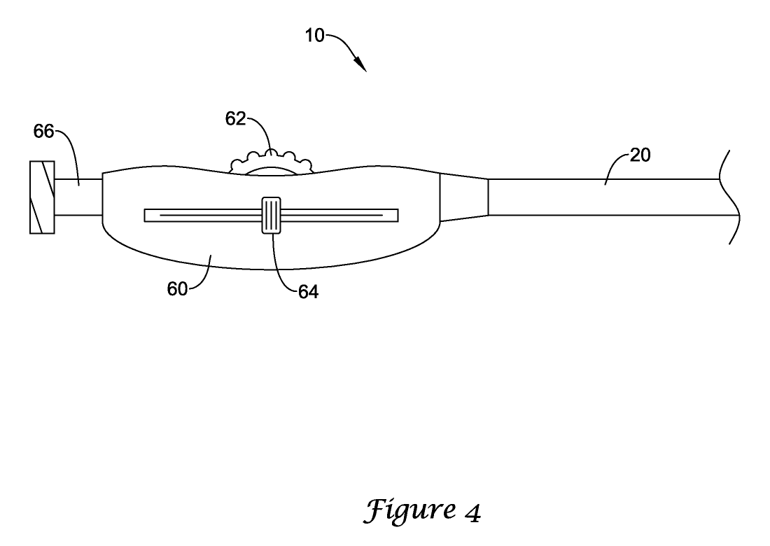

FIG. 1 is a side-view cross-section of an example snare instrument 10. The instrument 10 may include an elongated, flexible, tubular sheath 20, which may have a distal portion 21 and may terminate at a distal end 22. In some cases the sheath 20 may be configured to be used (e.g., advanced and retracted within) an endoscope, guide tube or other instrument. The sheath 20 may extend from the distal end 22 to a proximal end (not shown), which may attach to a handle 60 (not shown in FIG. 1, can be seen in FIG. 4) that can alter the position of the sheath 20. The distal end 22 of the sheath 20 may be open, thereby allowing other structures to exit the sheath 20 through its distal end 22.

A hollow shaft 30 may be disposed inside the tubular sheath 20. The hollow shaft 30 may have a distal portion 31 and may terminate at a distal end 32. The shaft 30 may be advanced and retracted with respect to the sheath 20. The shaft 30 may extend from the distal end 32 to a proximal end (not shown), which may also attach to the handle 60 that can alter the position the shaft 30 with respect to the sheath 20. In some cases, the same handle controls the positioning of both the sheath 20 and the shaft 30. In other cases, the sheath 20 and the shaft 30 are positioned by separate handles. In some cases, the handle or handles are hand-operated. In some cases, the sheath 20 may be stiffer than the shaft 30. In some cases, the hollow shaft 30 may have a closed distal end 32, although in other cases, the distal end 32 may be open. In some cases, the hollow shaft 30 may be or otherwise include a hypotube.

All or a portion of the hollow shaft 30 may be pre-formed to have a curvature in its relaxed state. In some cases, only the distal portion 31 of the shaft 30 is pre-formed to have a curvature. Because the hollow shaft 30 may be less stiff than the sheath 20, the hollow shaft 30 may take on the shape of the sheath 20 when the pre-curved portion is inside the sheath 20. If the sheath 20 is essentially straight, then the pre-curved portion of the hollow shaft 30 is essentially straight when inside the sheath 20. When the pre-curved portion of shaft 30 is extended out of the sheath 20, distally beyond the distal end 22 of the sheath 20, the pre-curved portion assumes its relaxed, curved state. In some cases, the curvature has a constant radius of curvature throughout. In other cases, the pre-formed portion has a bend, with less bent portions on one side or on both sides adjacent to the bend. Note that the hollow shaft 30 and/or the snare wire 40 may be pre-curved. These are just examples.

The hollow shaft 30 may have a longitudinal slit or slot 50 on its distal portion 31. The slit 50 may have distal end 52 near the distal end 32 of the shaft 30, and may not extend fully to the distal end 32 of the shaft 30. In other cases, the slit 50 extends fully to the distal end 32 of the shaft 30. In some cases, the slit 50 in the shaft 30 faces an interior of the curvature of the distal portion 31 of the shaft 30.

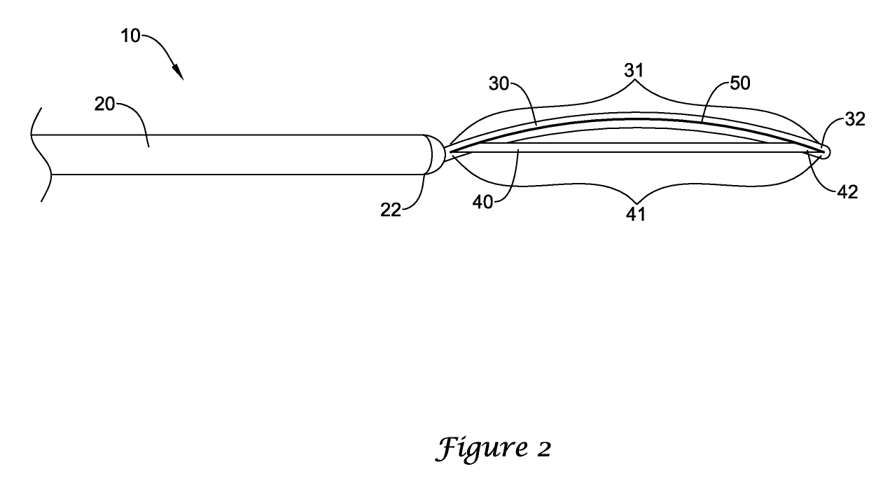

As seen in FIG. 2, a snare wire 40 may be disposed within the hollow shaft 30, may have a distal portion 41 and may terminate at a distal end 42. The snare wire 40 may be stiff enough so that it may be forced distally, or pushed, and may transfer force from its proximal end to its distal end. In some cases, the snare wire 40 may be less stiff than the hollow shaft 30, which in turn may be less stiff than the sheath 20. The snare wire 40 may be advanced and retracted with respect to the shaft 30, for example, by handle 60 at its proximal end. In some cases, the handle may be the same handle that controls the positions of the shaft 30 and/or sheath 20. In other cases, the wire 40, the shaft 30 and sheath 20 may be positioned by separate handles. The distal end 42 of the snare wire 40 may be attached to the shaft 30 at or near its closed distal end 32. In general, the deployment force for the snare may be controlled by the selection of suitable materials and/or the size of the snare wire relative to the slit 50.

Note that the snare wire 40 may have any suitable cross-section, including round, sharpened, serrated, triangular, multi-angular, square, and so forth. These are just examples.

It may be instructive to examine the snare instrument 10 in various stages of use. For example, prior to deployment, the snare instrument 10 may resemble the stage shown in FIG. 1, with the hollow shaft 30 contained within the sheath 20, the snare wire 40 contained within the hollow shaft 30 and not extending through the longitudinal slit 50 in the shaft 30, and the sheath 20, the shaft 30, and the snare wire 40 all being generally straight at their respective distal portions. During use, the snare instrument 10 may be advanced distally through the endoscope into a body lumen and may be positioned near a target area such as a polyp. At the end of the polypectomy, after the polyp has been severed, the snare instrument 10 may be withdrawn from the endoscope in this stage as well.

Once the snare instrument 10 is positioned near the polyp, the hollow shaft 30 may be distally advanced beyond the distal end 22 of the sheath 20, or, equivalently, the sheath 20 may be proximally retracted to expose the distal portion 31 of the hollow shaft 30. When the distal portion 31 of the hollow shaft 30 is released from the sheath 20, it may assume its pre-formed state and may become curved. This stage is shown in FIG. 2.

The longitudinal slit 50 at the distal portion 31 of the hollow shaft 30 may face the interior of the curvature. The snare wire 40, without any additional advancing or retracting, with respect to the hollow shaft 30, may take on the shape shown in FIG. 2. In other words, when the shaft 30 is positioned so that the distal portion 31 is disposed distally of the distal end 22 of the sheath, the snare wire 40 may extend out of the slit 50. The snare wire 40 may remain fastened to the shaft 30 at or near the distal end 52 of the longitudinal slit 50. Because the hollow shaft 30 may be stiffer than the snare wire 40, the snare wire 40 may take on a largely straight path from the distal end 52 of the slit 50, outside the hollow shaft 30, to the proximal end of the slit 50, and may then re-enter the hollow shaft 30 to extend toward the proximal end of the hollow shaft 30. In this stage, the curved distal portion 31 of the hollow shaft 30 and the relatively straight distal portion 41 of the snare wire 40 may form a loop.

Alternatively, the snare wire 40 may remain inside the hollow shaft 30 for the duration of the longitudinal slit 50. This may occur through careful sizing or orientation of the slit 50, such as making the width of the slit 50 comparable to an outer diameter of the snare wire 40, or by skewing the orientation of the slit 50 so that it does not face directly toward the interior of the curvature but is angled slightly away from the interior of the curvature. In these cases, friction may hold the snare wire 40 inside the hollow shaft 30 for the duration of the longitudinal slit 50.

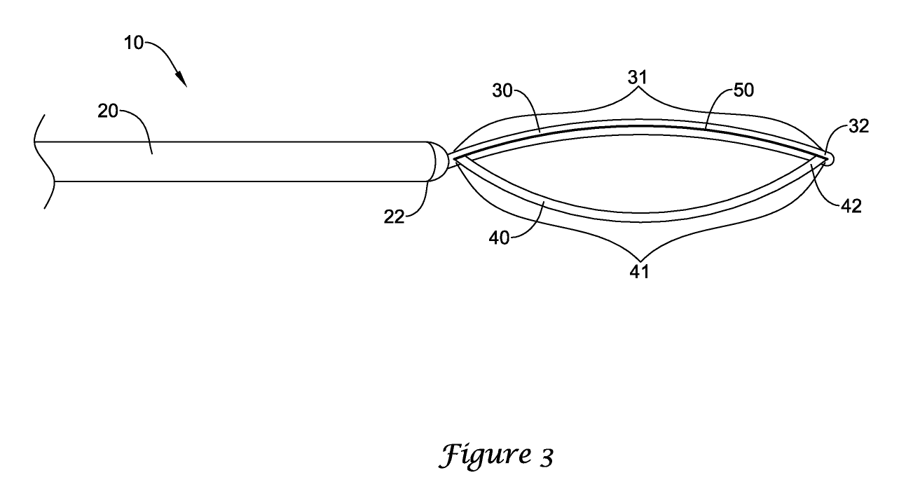

Once the distal portion 31 of the hollow shaft 30 is exposed and assumes its pre-curved state, the snare wire 40 may be forced distally from its proximal end, and may bow outward through the longitudinal slit 50. This stage is shown in FIG. 3. This stage may be referred to as a “deployed” stage. In this deployed stage, the curved distal portion 31 of the hollow shaft 30 and the bowed distal portion 41 of the snare wire 40 may form an oval-shaped loop. Shapes other than oval may include semi-circular, angular or other shapes advantageous to the procedure.

Note that there may be several ways of achieving the bowed state. In one case, the excess snare wire 40 may be pushed forward, so that it forces a bend that exits the shaft 30. In another case, the snare wire 40 may be pulled backwards to force the shaft 30 to bend. In still another case, the shaft 30 may be bent separately, forcing the snare wire 40 out. The above cases may be combined in any suitable manner, as well.

In the deployed stage, the practitioner places the loop over the polyp. The combination of the rounded distal portion 32 of the shaft 30 and the pre-formed curvature in the shaft 30 may aid in positioning the loop about the polyp. In addition, one or more of the surfaces of the shaft 30 and/or the wire 40 may be roughened or otherwise altered so as to improve the ability of the instrument 10 to “grip” the polyp. When properly positioned, the clinician may proximally retract the snare wire 40 and/or proximally retract the shaft 30 to sever the polyp and detach it from the anatomy. In some embodiments, cautery current may be passed down one or more of the shaft 30 and the snare wire 40 to cauterize the treatment site.

Once the polyp has been detached, the hollow shaft 30 and snare wire 40 may be retracted proximally into the distal portion 21 of the sheath 20. In some cases, such a retraction may be done all at once, going from the stage of FIG. 3 directly to the stage in FIG. 1. In other cases, the snare wire 40 may be retracted proximally first, which may reduce the outward bowing of the snare wire. In these cases, the outward bowing may be reduced or may be eliminated, as in the stage of FIG. 2. The hollow shaft 30 may be proximally retracted inside the sheath 20, or, equivalently, the sheath 20 may be distally advanced over the distal portion 31 of the hollow shaft 30. Once inside the sheath 20, the hollow shaft 30 loses its pre-formed curvature and assumes the shape of the sheath 20. The sheath 20, along with its interior contents, is then removed proximally from the endoscope.

It may be beneficial to refer to particular configurations for the snare instrument 10. A first configuration may be shown in FIG. 1, where the distal portion 31 of the hollow shaft 30 is inside the sheath 20. A second configuration may be shown in either FIG. 2 or FIG. 3, where the distal portion 31 of the hollow shaft 30 is advanced distally beyond the distal end 22 of the sheath 20 and assumes its pre-formed curvature.

In this second configuration, the snare wire 40 may be generally relaxed or forced proximally, as in the generally straight shape of FIG. 2, or may be forced distally, as in the outwardly bowed shape of FIG. 3.

In general, various handles may be used to actuate the device. For example, the device may use one or more spools that slide on a shaft, and so forth. FIG. 4 shows a generic hand-operated handle 60 at the proximal end of the snare instrument 10. Such a handle 60 may optionally include separate controls for positioning the snare wire 40 with respect to the hollow shaft 30 and for positioning the hollow shaft 30 with respect to the sheath 20. For example, a first handle member 62, which may take the form of a thumb wheel, may be coupled to the shaft 30 and may be used to move the shaft 30 proximally and distally. A second handle member 64, which may take the form of a slidable button, may be coupled to the snare wire 40 and may be used to move the wire 40 proximally and distally. In other embodiments, the handle members 62/64 may be switched so that the handle 62 is coupled to the snare wire 40 and the handle 64 is coupled to the shaft 30. In addition, or in the alternative, a proximal grasp handle 66 may also be included that may be coupled to the shaft 30 or the snare wire 40, which can be used to move either structure proximally and distally. These are just examples. Some or all of the controls 62/64/66 may include torsional control, so that the tip of the snare instrument 10 may be rotated as needed and placed over the polyp. In addition, the device may optionally include an electrical connector for energizing the snare wire.

The materials that can be used for the various components of the snare instrument 10 (and/or other sheaths, shafts or wires disclosed herein) may include those commonly associated with medical devices including metals, metal alloys, polymers, metal-polymer composites, ceramics, combinations thereof, and the like, or other suitable materials. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP. In addition, the device handle may optionally include one or soft grip portions, which may include a polymer such as TPE that may be molded onto the handle.

In at least some embodiments, portions or instruments 10 disclosed herein or the various components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of snare instrument 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of snare instrument 10 to achieve the same result.

In some embodiments, a degree of Magnetic Resonance Imaging (MRI) compatibility is imparted into snare instrument 10. For example, the instruments 10 and/or components thereof may be made of a material that does not substantially distort the image and create substantial artifacts (i.e., gaps in the image). Certain ferromagnetic materials, for example, may not be suitable because they may create artifacts in an MRI image. Snare wire 40 and/or the tubular members, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the exterior surface of the snare instrument 10 (including, for example, the exterior surface of snare wire 40 and/or the exterior surface of hollow shaft 30) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the sheath, or in embodiments without a sheath over portion of snare wire 40 and/or tubular member, or other portions of snare instrument 10. Alternatively, the sheath may comprise a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or sheath may be formed, for example, by coating, extrusion, co-extrusion, interrupted layer co-extrusion (ILC), gradient extrusion, or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

It should be noted that although a sheath 20 is illustrated in the figures, the sheath 20 itself may not be necessary. In some cases, the snare wire 40 may be delivered through a small diameter lumen, such as inside a needle or a small catheter, without a sheath. For instance, the snare wire 40 may be delivered through an endoscopic ultrasound-guided fine needle aspiration system. Alternatively, the snare wire 40 may be delivered through one of several commercially available small visualization catheters, such as SPYGLASS® direct visualization catheters commercially available from Boston Scientific.

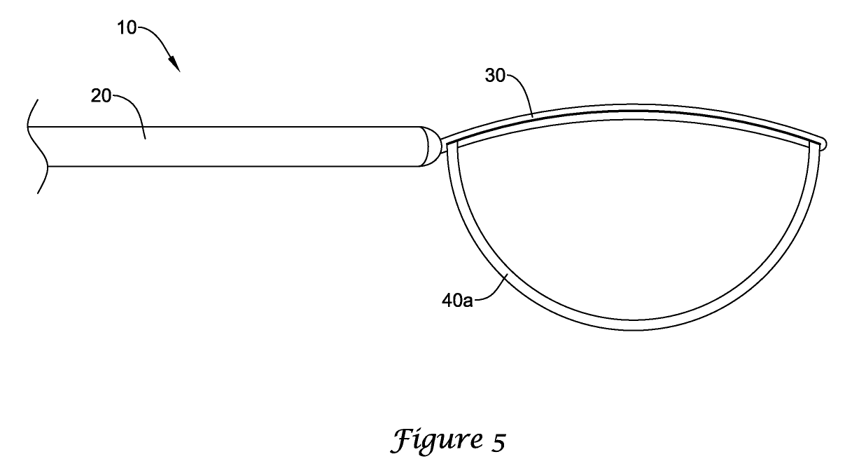

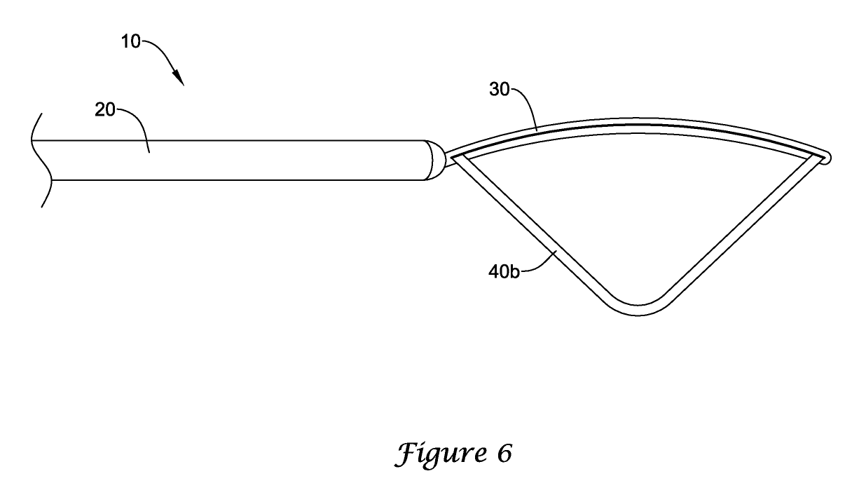

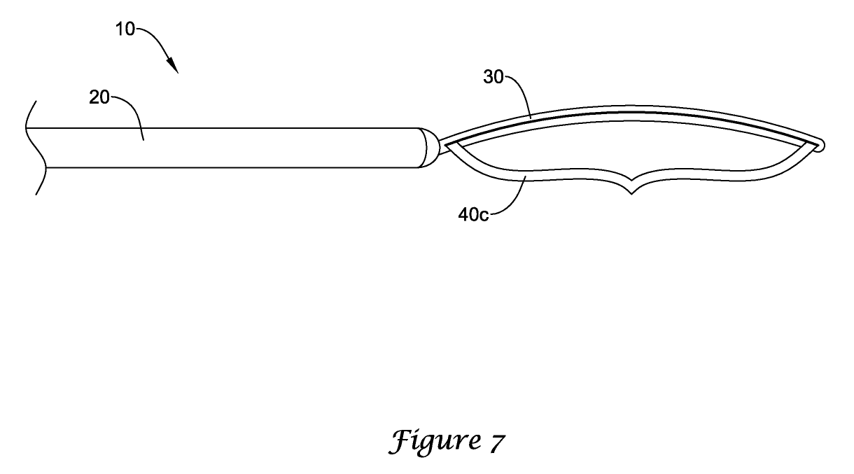

Note that the oval-shaped bow of the snare wire 40 is not the only shape available. Other example shapes are shown in FIGS. 5 through 7. FIG. 5 is a top view of an example snare having a snare wire 40a with a semicircular bow. FIG. 6 is a top view of an example snare having a snare wire 40b with an angled bow. FIG. 7 is a top view of an example snare having a snare wire 40c with a bracket-shaped bow. Other suitable shapes may be used as well.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A polypectomy snare instrument, comprising:
a hollow shaft, a distal portion of the shaft having a slit formed therein, the shaft being shiftable between a first configuration wherein the distal portion is substantially straight and a second configuration wherein the distal portion includes a bend; and
a snare wire disposed within the shaft, a distal section of the snare wire extending through the slit when the shaft is in the second configuration.

2. The polypectomy snare instrument of claim 1, further comprising an elongated sheath having a distal end, wherein the hollow shaft extends through and is movable relative to the sheath.

3. The polypectomy snare instrument of claim 2, wherein the shaft is in the first configuration when the distal portion of the shaft is disposed within the sheath.

4. The polypectomy snare instrument of claim 3, wherein the shaft shifts to the second configuration when the distal portion of the shaft is advanced distally of the distal end of the sheath.

5. The polypectomy snare instrument of claim 4, wherein the distal section of the snare wire and the distal portion of the shaft form a loop when the shaft is in the second configuration.

6. The polypectomy snare instrument of claim 4, wherein the distal section of the snare wire is disposed within the distal portion of the shaft when the shaft is in the first configuration.

7. The polypectomy snare instrument of claim 6, wherein the distal section of the snare wire extends out of the slit when the shaft is in the second configuration.

8. The polypectomy snare instrument of claim 2, wherein the snare wire is configured to be urged distally relative to the sheath.

9. The polypectomy snare instrument of claim 8, wherein the snare wire bows outward when urged distally.

10. The polypectomy snare instrument of claim 1, wherein the shaft includes a hypotube.

11. The polypectomy snare instrument of claim 1, further comprising a handle for shifting the shaft between the first configuration and the second configuration.

12. The polypectomy snare instrument of claim 11, wherein the handle includes a member for distally moving the snare wire relative to the shaft.

13. A polypectomy snare instrument, comprising:
a hollow shaft having a distal portion and terminating at a distal end, the distal portion of the shaft being pre-formed to have a curvature;
wherein the distal portion of the shaft has a slit formed therein; and
a flexible wire disposed within the shaft, the wire being controllably extendible through the longitudinal slit.

14. The polypectomy snare instrument of claim 13, further comprising an elongated flexible tubular sheath having a distal end, wherein the hollow shaft extends through and is movable relative to the sheath.

15. The polypectomy snare instrument of claim 14,
wherein the wire is configured to be urged distally relative to the sheath; and
wherein the wire bows outward when the wire is urged distally.

16. The polypectomy snare instrument of claim 14, wherein the shaft is configured to shift between a first configuration where the distal portion is disposed within the sheath and a second configuration where the distal portion is disposed distally of the distal end of the sheath.

17. The polypectomy snare instrument of claim 16, further comprising a handle for shifting the shaft between the first configuration and the second configuration.

18. The polypectomy snare instrument of claim 17, wherein the handle includes a member for distally moving the snare wire relative to the shaft.

19. A method of performing a polypectomy, comprising:
providing a snare device, the snare device comprising:
an elongated sheath having a distal end,
a hollow shaft extending through and movable relative to the sheath, a distal portion of the shaft having a slit formed therein, the shaft being shiftable between a first configuration wherein the distal portion is substantially straight and a second configuration wherein the distal portion includes a bend, and
a snare wire disposed within the shaft, a distal section of the snare wire extending through the slit when the shaft is in the second configuration;
advancing the snare device through a body lumen to a position adjacent to a polyp;
distally advancing the shaft relative to the sheath to shift the shaft from the first configuration to the second configuration;
distally advancing the snare wire relative to the shaft to bow the distal section of the snare wire and form a loop with the shaft; and
disposing the loop about the polyp.

20. The method of claim 19, wherein the shaft advancing and retracting is controlled from a hand-operated handle disposed at a proximal end of the shaft.
